(19) United States
(12) Patent Application Publication
(10) Pub. No.: US 2021/0295991 A1
(43) Pub. Date: Sep. 23, 2021

(54) SYSTEM AND METHOD FOR IDENTIFYING HEALTHCARE ISSUES

(71) Applicant: SAFE & RELIABLE HEALTHCARE LLC, Evergreen, CO (US)

(72) Inventors: Allan S. FRANKEL, Acton, MA (US); Michael W. LEONARD, Evergreen, CO (US); Terri CHRISTENSEN FRANKEL, Acton, MA (US); Joshua PROULX, Lehi, UT (US); Daniel Joseph CRAMER, Murray, UT (US); John D. DUCE, Lehi, UT (US); Jared GOODEY, Evergreen, CO (US); Chad LANENGA, Evergreen, CO (US)

(73) Assignee: SAFE & RELIABLE HEALTHCARE LLC, Evergreen, CO (US)

(21) Appl. No.: 17/224,960

(22) Filed: Apr. 7, 2021

Related U.S. Application Data
(63) Continuation-in-part of application No. 15/686,092, filed on Aug. 24, 2017.
(60) Provisional application No. 62/379,181, filed on Aug. 24, 2016.
(60) Provisional application No. 62/505,068, filed on May 11, 2017.

(51) Int. Cl. G16H 40/63; G16H 10/60

(52) U.S. Cl. CPC G16H 40/63; G16H 10/60

(57) ABSTRACT

Identification and monitoring of healthcare issues includes determining how to process input data based on at least one of identity of a user device that transmits the input data to the system and categorization of the input data. A new record is created in a database of healthcare issues in a healthcare facility where the user device is located when the input data is received. Anonymous reporting of issues is supported by storing an identifier of the user device that transmits the input data to the system, so that the anonymous reporter can be invited by a manager to join in an anonymous chat session. Recording of data during supervisor walkrounds is supported by setting up scheduled walkrounds with default labels to simplify input of identified healthcare issues, while allowing for custom labels to be entered when needed.

### CROSS-REFERENCE TO RELATED APPLICATIONS

This application is a continuation-in-part of U.S. patent application Ser. No. 15/686,092, filed Aug. 24, 2017, which claimed the benefit under 35 U.S.C. § 119(e) of U.S. Provisional Patent Application Nos. 62/379,181, filed Aug. 24, 2016, and 62/505,068, filed May 11, 2017, all of which are hereby incorporated by reference in their entirety.

### BACKGROUND

The application relates to systems that allow for communication between group members and, more particularly, to electronic systems that allow for those communications, especially within work environments, such as hospitals.

Of particular concern in hospitals and other healthcare facilities is the identification and monitoring of healthcare issues. Known systems lack provisions for engaging with anonymous reporters of healthcare issues and robust support for easily recording healthcare issues during walkrounds by a supervisor.

### SUMMARY

An aspect of the method and system described below is determining how to process input data based on at least one of identity of a user device that transmits the input data to the system and categorization of the input data, and creating a new record in a database of healthcare issues in a healthcare facility where the user device is located when the input data is received. According to this aspect, the new record contains the input data and at least one of an identifier associated with the user device and a label associated with the input data.

An aspect of the method and system described below is to determine whether the database has stored the identifier associated with the user device prior to creation of the new record. Based on this determination, the system stores a user identifier when the identifier associated with the user device was stored in the database prior to creation of the new record and stores the identifier associated with the user device without any user identifier when the identifier associated with the user device was not stored in the database prior to the creation of the new record.

An aspect of the method and system described below is to send comments on the healthcare issue indicated by the input data to the user device only when the identifier associated with the user device was stored in the database prior to the creation of the new record.

An aspect of the method and system described below is to send a request for communication regarding the healthcare issue to the user device when the identifier associated with the user device was not stored in the database prior to the creation of the new record. The request for communication regarding the healthcare issue may include a link to an anonymous chat space.

An aspect of the method and system described below is to decide whether to schedule a new walkround by a supervisor, start a scheduled walkround by the supervisor, or view a selected previous walkround by the supervisor. When the new walkround is to be scheduled, walkround details are stored in the database, including at least one of a name of the new walkround, frequency of occurrence of the new walkround, a time of the new walkround, default notes, default participants, default location, and default labels for healthcare issues identified during the new walkround.

An aspect of the method and system described below is that after the walkround is started, how to process the input data is determined by inquiring of the supervisor whether to use default labels, or to receive at least one custom label, for a healthcare issue identified during the scheduled walkround.

An aspect of the method and system described below is that when default labels are selected by the supervisor, the new record stores an issue name and at least one identifier of a team member related to the healthcare issue identified during the scheduled walkround.

An aspect of the method and system described below is that when at least one custom label is selected by the supervisor, the new record stores the at least one custom label, an issue name and at least one identifier of a team member related to the healthcare issue identified during the scheduled walkround.

An aspect of the method and system described below is that after the selected previous walkround is selected by the supervisor, notes about at least one of the identified healthcare issues in the database having data recorded during the selected previous walkround and current status of the at least one of the identified healthcare issues are output on a display.

Additional features and advantages of the present invention are described in, and will be apparent from, the detailed description of the presently preferred embodiments and from the drawings.

### BRIEF DESCRIPTION OF THE DRAWINGS

These and other aspects and advantages will become more apparent and more readily appreciated from the following description of the exemplary embodiments, taken in conjunction with the accompanying drawings of which:

FIG. 1 is a schematic diagram of a system for learning, in accordance with an aspect of the invention;

FIG. 2 is a functional block diagram of software modules that execute in a processor of the system in FIG. 1;

FIG. 3A-3E are depictions of user interfaces produced by a display in the system in FIG. 1;

FIGS. 4A-4C are schematic front views of a user device in the system illustrated in FIG. 1;

FIG. 5 is a flowchart of a method for providing a learning environment;

FIG. 6 is a flowchart of another method for providing a learning environment;

FIG. 7 is a schematic diagram of an exemplary computer environment of the system in FIG. 1;

FIG. 8 is a flowchart of the reply to reporter feature;

FIG. 9 is a data structure diagram for data used by the reply to reporter feature;

FIG. 10 is a flowchart of the walkrounds feature; and

FIG. 11 is a data structure diagram for the walkrounds feature.

### DETAILED DESCRIPTION

Reference will now be made in detail to the preferred embodiments, examples of which are illustrated in the accompanying drawings, wherein like reference numerals refer to like elements throughout.

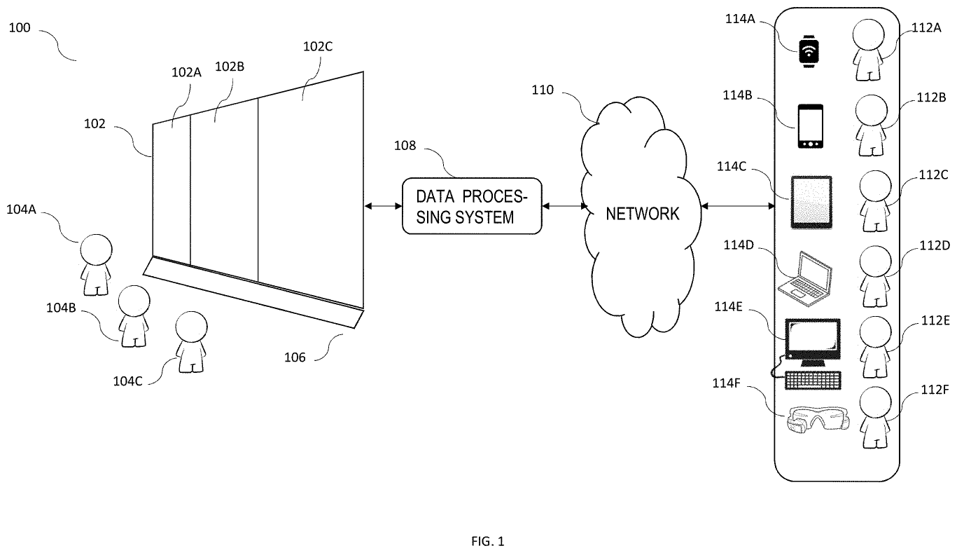

FIG. 1 illustrates a system 100 for processing healthcare data. The system 100 includes a group display 102, input equipment 106 provided in conjunction with the group display 102, a data processing system 108 and user input devices 114A-114F (collectively referred to as input devices 114) which are connected to data processing system 108 via a network 110.

The group display 102 may be subdivided into multiple display areas 102A, 102B, or 102C. Different display areas may be configured to display different information. For example, area 102A may display “New identified Issues”, area 102B may display “Resolved Issues” and area 102C may display “Pending Issues”. It may be appreciated by a person having ordinary skill in the art that the display 102 may be divided into further display areas or more than three display areas. The group display 102 may be any known display device capable of being viewed by a group of people, including a projector and screen, an (organic) light emitting diode (LED, OLED or AMOLED) display, a liquid crystal display (LCD), a plasma display, or a cathode ray tube (CRT) display. The group display 102 may be made up of a single display screen or multiple display screens may display portions that a viewer perceives as a single combined display.

The input equipment 106 may include a keyboard, a stylus, a mouse, a trackball, a touch screen, etc. The input equipment 106 may be used by local users 104A-1040 within the vicinity of the group display 102, to interact with the data processing system 108 by inputting and accessing information in the data processing system 108 for display on the group display 102.

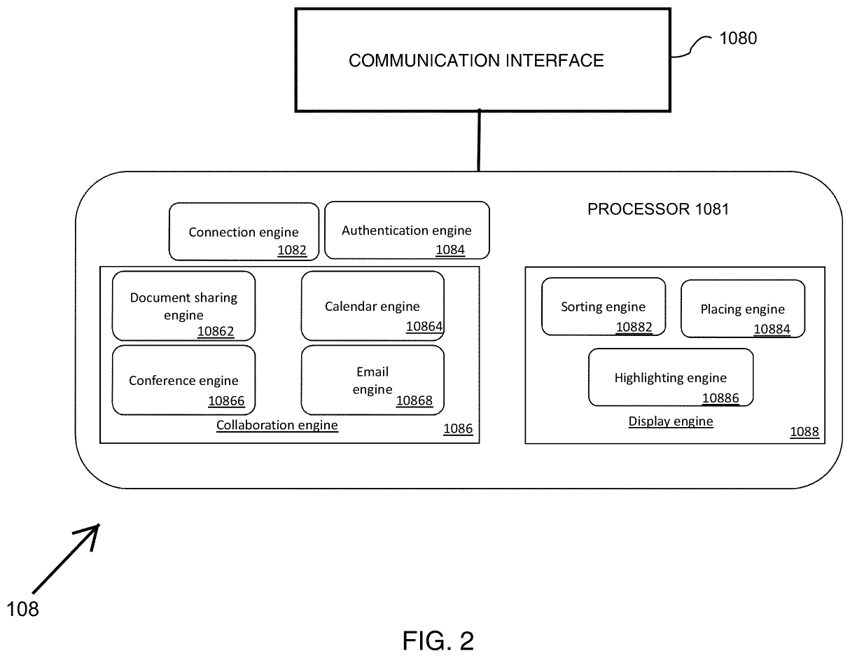

As illustrated in FIG. 2, the data processing system 108 may include at least one hardware processor 1081 executing a computer program to respond to user inputs and system-generated requests. The processor(s) 1081 may include a microprocessor, embedded or secure processors.

The network 110 may be a wired or a wireless network. The network 110 may be Local Area Network (LAN) that may be implemented using a TCP/IP network and may implement voice or multimedia over Internet Protocol (IP) using a Session Initiation Protocol (SIP). As illustrated in FIG. 2, processor 1080 in the data processing system 108 may be coupled to the display 102 and the input devices 114 via a communication interface 1080. The communication interface may employ connection protocols including, without limitation, direct connect, Ethernet (e.g., twisted pair 10/100/1000 Base T), transmission control protocol/internet protocol (TCP/IP), token ring, IEEE 802.11a/b/g/n/ac/x, etc. The network 110 may include, without limitation, a direct interconnection, local area network (LAN), wide area network (WAN), wireless network (e.g., using Wireless Application Protocol), the Internet, etc. Using the communication interface 1080 and the network 110, the data processing system 108 may communicate with the input devices 114. These input devices 114 may include, without limitation, personal computer(s), smart watches, tablet computer, desktop PCs, head mounted wearables, and various mobile devices such as cellular telephones, smartphones (e.g., Apple iPhone, Android-based phones, etc.) or the like. Each of the user input devices 114 corresponds to at least one of plural remote users 112A-112F (generally referred to as remote user 112).

In some embodiments, the processor 1080 may have access to one or more memory devices (not shown in figure) via a storage interface (also not shown in figure). The storage interface may connect to memory devices including, without limitation, memory drives, removable disc drives, etc., employing connection protocols such as serial advanced technology attachment (SATA), integrated drive electronics (IDE), IEEE-1394, universal serial bus (USB), fiber channel, small computer systems interface (SCSI), etc. The memory drives may further include a drum, magnetic disk drive, magneto-optical drive, optical drive, redundant array of independent discs (RAID), solid-state memory devices, solid-state drives, etc.

The data processing system 108 receives inputs from the connected input devices 114. As described above, each of the user input devices 114 corresponds to one of the remote users 112. Users may have various defined accessibility roles. Some users may have read access, some may have write access, and some may have both read and write access. The remote users 112 logon using their respective input devices 114. The users may add information through their respective input devices 114. The data processing system 108, when information is received via the network 110, analyzes the information and processes it to be displayed on the group display 102 on any of the display areas 102A, 102B, and 102C. The local users 104A, 104B, and 104C (collectively referred to as remote users 104) are those users who may be in the vicinity of the group display 102. They may cumulatively logon to the system 100 using input equipment 106. Any information added by any type of user, be it local users 104 or remote users 112, is analyzed and processed by the processor 1080 to be displayed on designated display areas 102A, 102B, and 102C.

The processor decides display areas based on the context of the information received or input received from the users 112, and 104. Also, the data processing system 108 provides collaboration tools, such as conference calls, including audio and video conference calls, document sharing, document editing, document collaborating, etc. By way of an example the local users 104 in the vicinity of group display 102 are able to collaborate or have a team huddle with the remote users 112. The remote users 112 thus may attend or collaborate in every team meeting or documents. Also, the remote users 112 and the local users 104 may be part of same team and may work together, even when not being present physically together, to identify key issues, problems, news, techniques, etc.

FIG. 2 illustrates software modules that may be executed by the processor 1080 in data processing system 108 which enables collaboration of remote users 112 and local users 104 in the learning system 100. The software modules executed by processor 1080 include a connection engine 1082, an authentication engine 1084, a collaboration engine 1086, and a display engine 1088.

The connection engine 1082, acts as a network interface and helps in connecting to the network 110. The connection engine may be either a microprocessor or a combination of multiple processors. Microprocessors may be either hardware or software compilations.

The authentication engine 1084, connected to the connection engine 1082, helps in identification and authentication of user logging in. The authentication engine 1082 helps to keep a check on user access of the system 100. The authentication engine 1082 may be coupled to a memory or a database to store the authentication details and verify the same. Also, the database may include information like which user may be provided what kind of access. The access level may be based on user hierarchy, seniority, user type, etc. The access may be read access, write access or a combination of both.

The collaboration engine 1086 helps users 112 and 104 to collaborate with each other. The collaboration engine 1086 may include a document sharing engine 10862, a calendar engine 10864, a conference engine 10866 and an email engine 10868.

The document sharing engine 10862 helps users to share and collaborate on various documents. The documents may be text files, images, videos, presentations spreadsheets, etc. the document sharing engine 10862 helps in identifying user who edits or accesses the document supported by time stamp of when the document was used. In this manner, the document sharing engine 10862 is configured to trace back to a user who makes changes or uses it and when.

Also, in case a user wrongly accesses a document, the document sharing engine may stop the user by providing a warning to the user on his respective device. For this functionality, the document sharing engine 10862 may be connected to the memory to access the document sharing permissions, etc. In an implementation, the documents sharing engine may have its own cache memory for the same functioning. Sharing of documents may be done by providing access through either user IDs of the users or email addresses. Further, the documents sharing engine 10862 may have another setting of sharing a particular document with all users that is keep the document public. Documents shared may also include report of errors, or single error reported.

The calendar engine 10864, provides meeting possibilities to users 112 and 104. The calendar engine collaborates calendars of all users 112 and 104 and may initiate meeting sessions based on events found. The events may be calendar invites or calendar events received for a specific time and date. A user who wants to have a meeting may send in calendar invites to other user desired on the meeting. When these users accept the invites, the events are added on the respective user's device calendar. When meeting time approaches, user is intimated about the same. In other implementation, the meeting may automatically start on specific time or in some buffer time.

The conference engine 10866 helps users 112 and 104 to have conference calls. The conference calls may be either audio calls, video calls or a combination thereof. Conference calls may be made by inviting participants using their email IDs or user IDs.

The email engine 10868 may also assist in initiating meetings between users 112 and 104, similar to the calendar engine 10864. The email engine 10868 may be able to automatically pick up meeting request from mails received by a user. This may be done using a semantic analysis. Also, when a user sends a meeting invite, the email engine may automatically create a meeting event. Also, the email agent 10868 may be able to send reminders to invited users before the meeting event happens.

The display engine 1088 is responsible for displaying the right content at the right place at the right time. The display engine is coupled with the authentication engine 1084. The display engine receives data from users 112 and 104 after being authenticated and displays the same at right place on the group display 102. The display engine 1088 may include a sorting engine 10882, a placing engine 10884 and a highlighting engine 10886.

The sorting engine 10882 helps in sorting data received as per the content written. For example, the data from a user about an error is sorted out to be an error information. Hence, it may be tagged as an error information and sorted out to be placed on specific area “Issues identified” 102A. This may be done using a semantic analysis by the sorting engine 10882.

The placing engine 10884 decides positioning of the information received, as per the tag of the information sorted by the sorting engine 10882. The placing engine 10882 then may also display name of user adding information and time of the information received. For placing of the information, the placing engine 10884, may maintain a repository of tags and their placing on the group display 102, based on which placing of the text is performed.

The highlighting engine 10886 may be used by a user to highlight a certain severe pending issue or a rectified issue. Various types of highlighting colors may be used to convey different information. The highlighting engine 10886 may have this functionality specially to help users having read access only. In this manner, users with only read access may be able to convey information without editing the same.

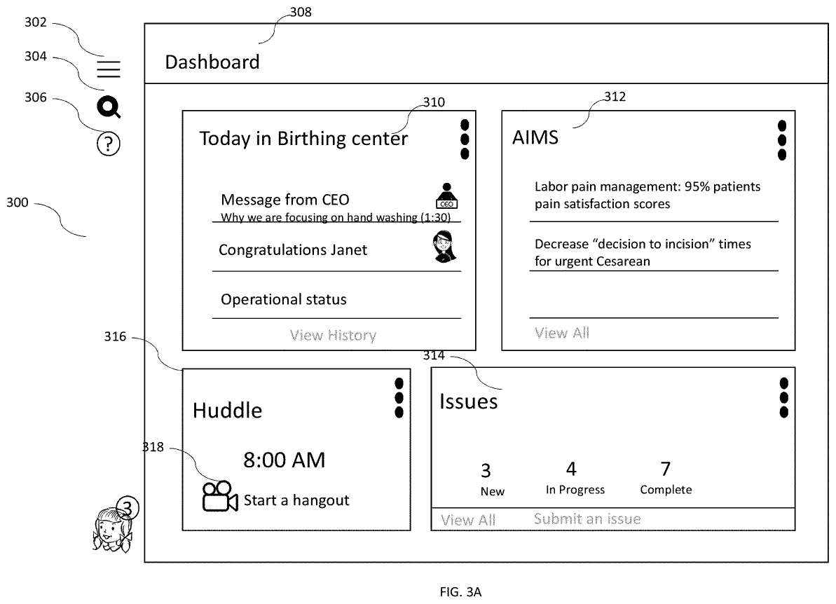

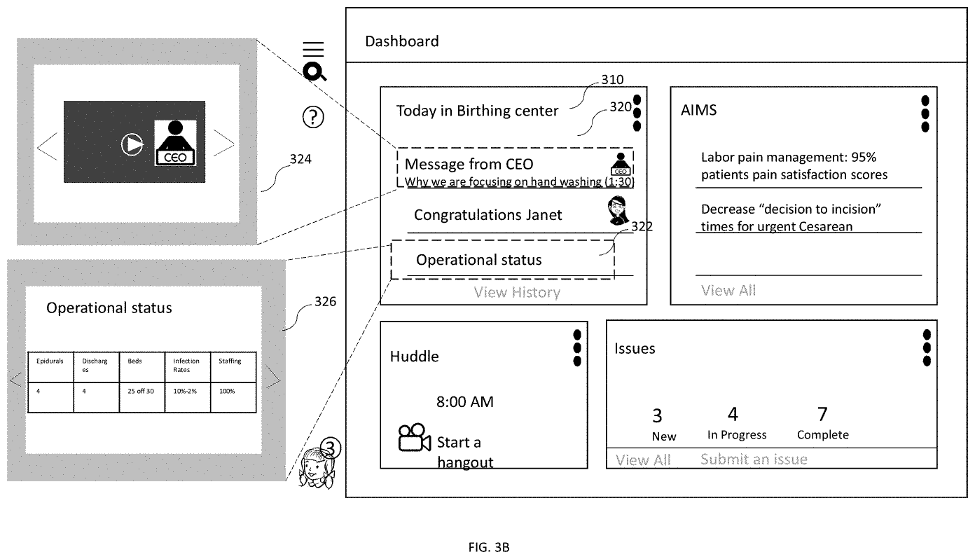

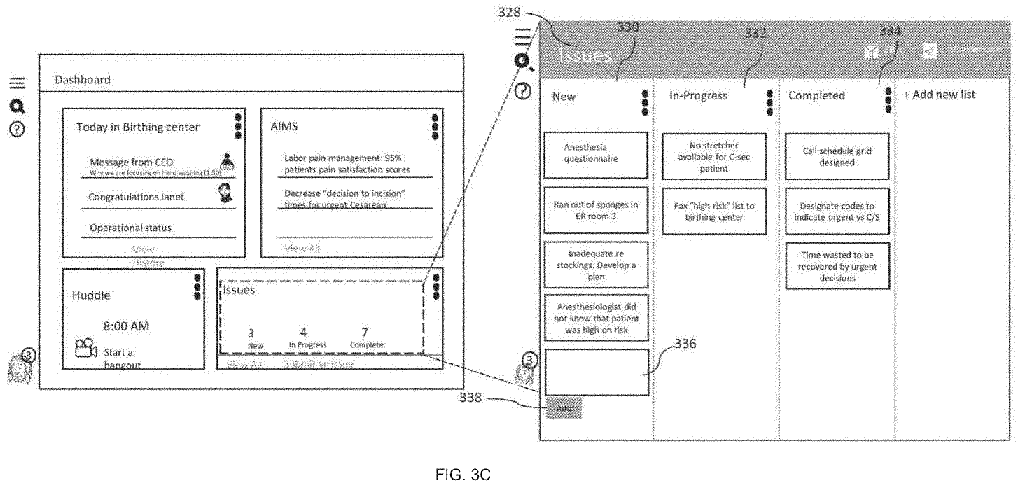

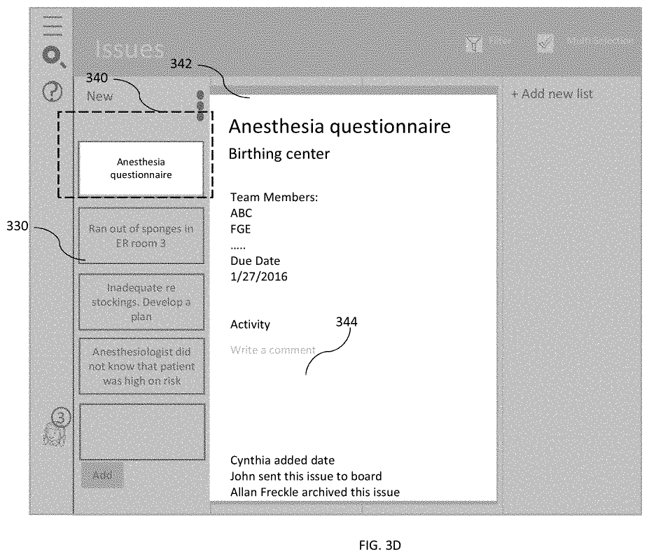

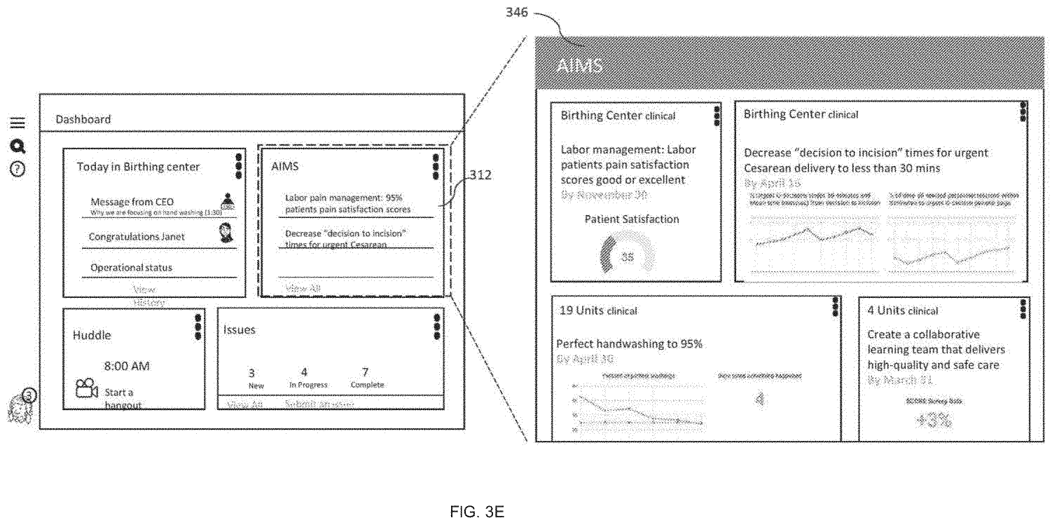

Now referring to FIG. 3A-3F, various user interfaces at various steps and level of information are illustrated, in accordance with an aspect of the invention. FIG. 3A depicts a user interface 300 after the user logs on to the system 100. The user interface 300 has functionality similar to that provided by the group display 102 and capabilities described for one apply to the other. The user interface 300 may depict a first landing page after the user logs in. The user interface 300 may include a menu input 302. The menu input 302 may be used by a user to access various functions of the user interface 300. The user interface 300 may also include a search input tab 304. The search tab 304 may be utilized to search for various information like issues pending, etc. Furthermore, the user interface may utilize a help tab 306. The help tab 306 may be utilized to help a user or provide to the user various information about the user interface 300.

Furthermore, the user interface 300 may include a dashboard 308 that may be further divided into multiple information areas to display multiple types of information. The user interface 300 may include a general information area 310, an aims area 312, and issues area 314. The general information area 310 provides general important information like “A message from the CEO”, or “operational status of a new wing”, etc. This information may generally be information to public at large and not for some specific users. The aims area 312 helps providing information to users about specific aims that their organization is working towards. It may involve information about decreasing a particular type of a problem faced by users, and may get updated regularly or in real-time. The issues area 314 may contain information about new issues identified, in progress resolution of issues and also completely rectified issues.

The user interface 300, may also include an information input area 316 wherein a user may enter information of interest or use. For example, a user may input information about issues being faced in a process, etc. which, after sorting by the sorting engine 10882, may display it in the issues section 314. The user interface may also include a tab or icon 318 to initiate collaboration, e.g., via Google hangout. User can simply click on the tab 318 to initiate a meeting with the users invited or a general meeting with all users.

Now referring to FIG. 3B, there is shown second level information that is available to users accessing the general information area 310. A user may be able to access more information when he touches the messages on the general information area 310. For example, a user may touch a message 320, i.e., “A message from the CEO”, or message 322, i.e., “Operational status” to see more information about the same. The messages may open a pop-up window or a completely different window 324 and 326 respectively, to display additional information.

Similarly, as depicted in FIG. 3C, the user may touch the issues identified at 314. This input from the user may provide a new sub-user interface 328. The sub-user interface 328 may have further bifurcation of display area into multiple parts displaying New issues identified 330, In progress issues 332, and Completed issues 334 areas. User may add a new issue or in progress issue or completed issue information through an information addition area 336 and hitting a submit button 338. Further, the user may get into third level of information wherein, user going through the new issues identified area 330 may want to have more information about a message 340 as depicted in FIG. 3D. The user may give his/her input by touching the message 340 that may open a pop-up window 342 that may include an activity enter area 344 to add some notes about the message that may act as supporting information.

Referring to FIG. 3E, a user wanting to know about information in Aims area 312, may provide input by touching the area. This action may open up a new window 346 with a description of Aims area 312.

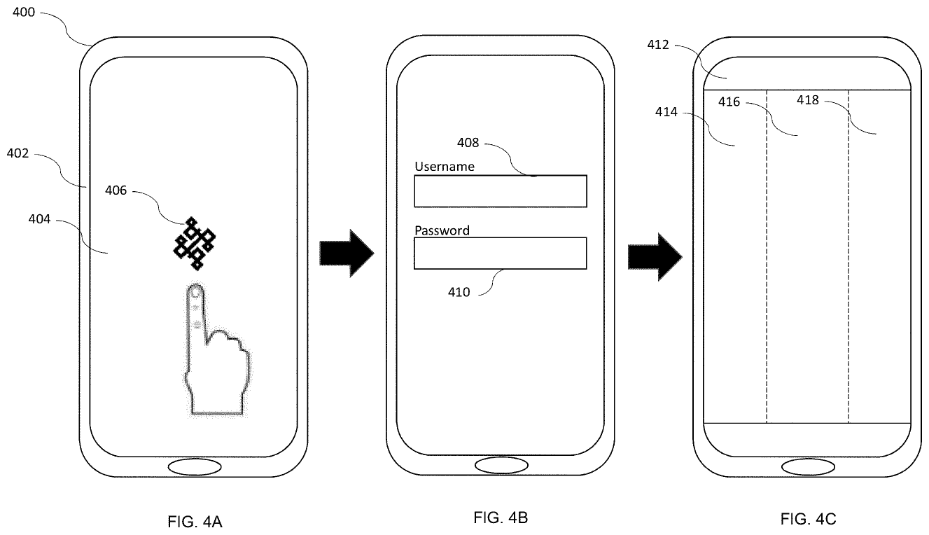

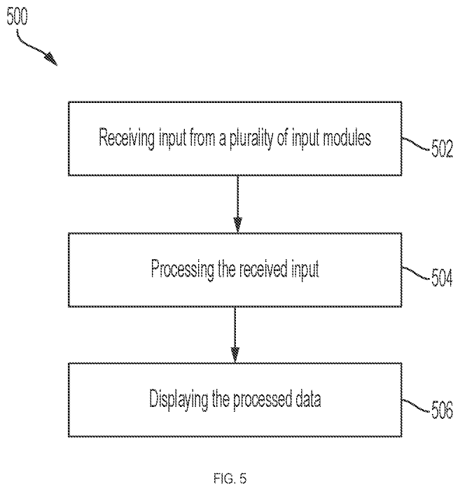

Referring to FIG. 4, a user interface 412, is provided on a user device 400 having a display frame 402 and a display unit 404. The user interface 412 may be accessed using a software application 406 provided on the user device 400. The software application may be initiated by user's touch input. After this input, the software application may display an authentication interface or login interface having a username field 408 and a password field 410. User may also be able to login using his other authentication details like fingerprint, or biometric scan or voice prints, etc. After successful log in, user is displayed with the user interface 412 that may include multiple information display areas 414, 416 and 418.

FIG. 5 illustrates a method 500 for learning in accordance with another aspect of the invention. At step 502, the data processing system 108, receives inputs from the user input devices 114 corresponding to the plural users 112. As described, the user input devices 114 may have the software application 406 which when activated by a user may authenticate the user by requesting the user of his username and password. The user may then input information which when received by the data processing system 108 is processed by the processor 1080. The processing includes semantic analysis, and further analysis as to which part of the group display 102 should the information be displayed on is performed. Further, at step 506, the processed data is then displayed in the relevant part of the group display 102.

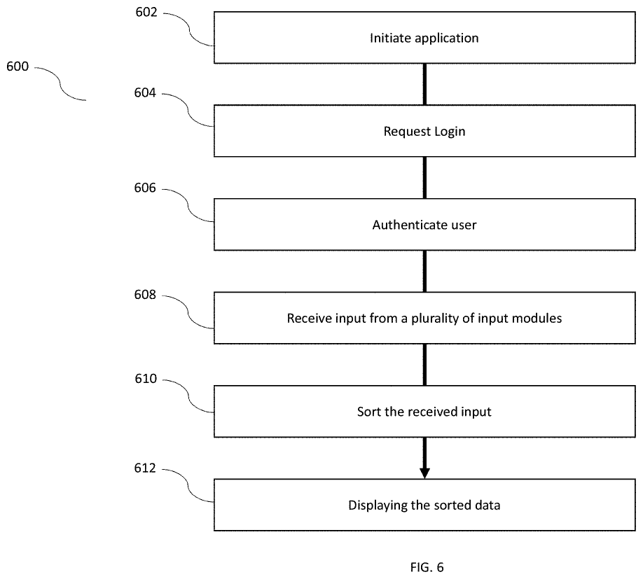

FIG. 6 illustrates a method 600 in accordance with another aspect of the invention. At step 602, a user may initiate the application for connecting her user input device 114 to the data processing system 108 for display of data on group display 102. The application may be a software application embedded into the Operating Software or may be downloaded and installed via an application store. The application when initiated, at step 604, provides an authentication interface to the user. the user may enter his log in credentials to log in. Log in may be through a username password, or other log in credentials like fingerprint, biometric scans, etc. At step 606, the processor 1080 checks the log in credentials and verifies it from the memory or database. After successful login, at step 608, the processor 1080 receives input from the user and identifies the information being input. Further, at step 610, the processor 1080 sorts the data based on the context of the information added. The context may be extracted using semantic analysis. Further, at step 612, as per the sorted data, a specific area of the group display 102 is identified where the data is to be displayed.

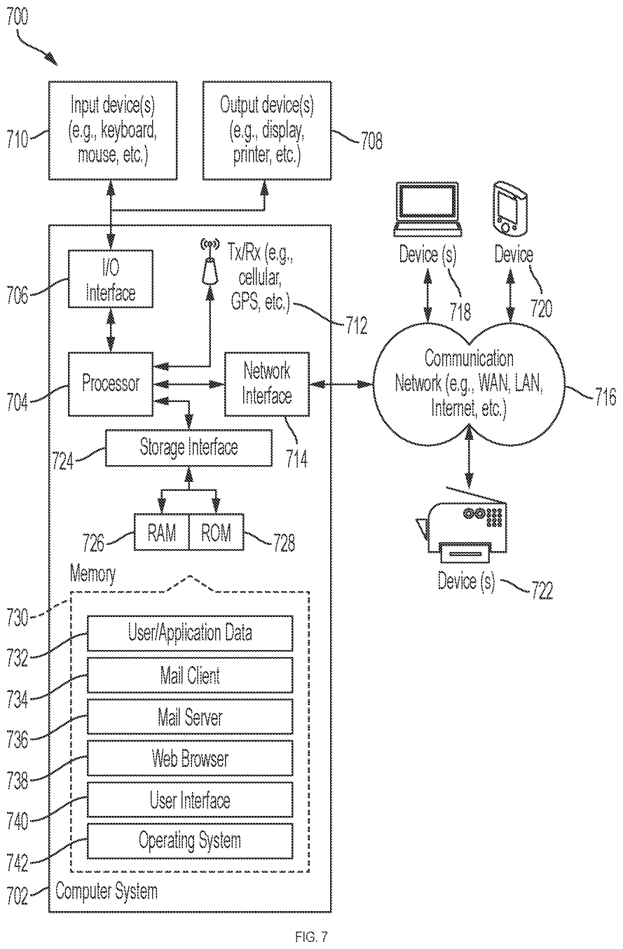

In addition to the components illustrated in FIG. 2, data processing system 108 may include an exemplary computer system 702, illustrated in FIG. 7. Computer system 702 may have a central processing unit (CPU) or hardware processor 704. Processor 704 may include at least one hardware processor for executing program components that respond to user- or system-generated requests. A request may be generated by a person using any of the input devices described above, or by such a device automatically. Processor 704 may include specialized processing units such as integrated system (bus) controllers, memory management control units, floating point units, graphics processing units, digital signal processing units, etc. Processor 704 may include a microprocessor, such as AMD Athlon or Sempron, Duron or Opteron, ARM's application, embedded or secure processors, IBM PowerPC, Intel's Core, Itanium, Xeon, Celeron or other line of processors, etc. Processor 704 may be implemented using mainframe, distributed processor, multi-core, parallel, grid, or other architectures. Some embodiments may utilize embedded technologies like application-specific integrated circuits (ASICs), digital signal processors (DSPs), Field Programmable Gate Arrays (FPGAs), etc.

Processor 704 may communicate with one or more input/output (I/O) devices via an I/O interface 706. I/O interface 706 may employ communication protocols/methods such as, without limitation, audio, analog, digital, monaural, RCA, stereo, IEEE-1394, serial bus, universal serial bus (USB), infrared, PS/2, BNC, coaxial, component, composite, digital visual interface (DVI), high-definition multimedia interface (HDMI), RF antennas, S-Video, VGA, IEEE 802.11 a/b/g/n/ac/x, Bluetooth, cellular (e.g., code-division multiple access (CDMA), high-speed packet access (HSPA+), global system for mobile communications (GSM), long-term evolution (LTE), WiMAX, or the like), etc.

Using I/O interface 706, computer system 702 may communicate with one or more I/O devices. For example, an input device 708 may be an antenna, keyboard, mouse, joystick, (infrared) remote control, camera, card reader, fax machine, dongle, biometric reader, microphone, touch screen, touchpad, trackball, sensor (e.g., accelerometer, light sensor, GPS, gyroscope, proximity sensor, or the like), stylus, scanner, storage device, transceiver, video device/source, visors, etc. An output device 710 may be a printer, fax machine, video display (e.g., cathode ray tube (CRT), liquid crystal display (LCD), light-emitting diode (LED), plasma, or the like), audio speaker, etc. In some embodiments, a transceiver 712 may be coupled to processor 704. Transceiver 712 may facilitate various types of wireless transmission or reception. For example, transceiver 712 may include an antenna operatively connected to a transceiver chip (e.g., Texas Instruments WiLink WL1283, Broadcom BCM4760IUB8, Infineon Technologies X-Gold 618-PMB9800, or the like), providing IEEE 802.11a/b/g/n/ac/x, Bluetooth, FM, global positioning system (GPS), 2G/3G HSDPA/HSUPA communications, etc.

In some embodiments, processor 704 may communicate with a communication network 714 via a network interface 716. Network interface 716 may communicate with communication network 714. Network interface 716 may employ connection protocols including, without limitation, direct connect, Ethernet (e.g., twisted pair 10/100/1000 Base T), transmission control protocol/internet protocol (TCP/IP), token ring, IEEE 802.11a/b/g/n/ac/x, etc. Communication network 714 may include, without limitation, a direct interconnection, local area network (LAN), wide area network (WAN), wireless network (e.g., using Wireless Application Protocol), the Internet, etc. Using network interface 716 and communication network 714, computer system 702 may communicate with devices 718, 720, and 722. These devices may include, without limitation, personal computer(s), server(s), fax machines, printers, scanners, various mobile devices such as cellular telephones, smartphones (e.g., Apple iPhone, Android-based phones, etc.), tablet computers, eBook readers (Amazon Kindle, Nook, etc.), laptop computers, notebooks, gaming consoles (Microsoft Xbox, Nintendo DS, Sony PlayStation, etc.), or the like. In some embodiments, the computer system 602 may itself embody one or more of these devices.

In some embodiments, processor 704 may communicate with one or more memory devices (e.g., a RAM 726, a ROM 728, etc.) via a storage interface 724. Storage interface 724 may connect to memory devices 730 including, without limitation, memory drives, removable disc drives, etc., employing connection protocols such as serial advanced technology attachment (SATA), integrated drive electronics (IDE), IEEE-1394, universal serial bus (USB), fiber channel, small computer systems interface (SCSI), etc. The memory drives may further include a drum, magnetic disc drive, magneto-optical drive, optical drive, redundant array of independent discs (RAID), solid-state memory devices, solid-state drives, etc.

Memory devices 730 may store a collection of program or database components, including, without limitation, an operating system 732, a user interface application 734, a web browser 736, a mail server 738, a mail client 740, a user/application data 742 (e.g., any data variables or data records discussed in this disclosure), etc. Operating system 732 may facilitate resource management and operation of computer system 702. Examples of operating system 732 include, without limitation, Apple Macintosh OS X, Unix, Unix-like system distributions (e.g., Berkeley Software Distribution (BSD), FreeBSD, NetBSD, OpenBSD, etc.), Linux distributions (e.g., Red Hat, Ubuntu, Kubuntu, etc.), IBM OS/2, Microsoft Windows (XP, Vista/7/8/10, etc.), Apple iOS, Google Android, or the like.

User interface 734 may facilitate display, execution, interaction, manipulation, or operation of program components through textual or graphical facilities. For example, user interfaces may provide computer interaction interface elements on a display system operatively connected to computer system 702, such as cursors, icons, check boxes, menus, scrollers, windows, widgets, etc. Graphical user interfaces (GUIs) may be employed, including, without limitation, Apple Macintosh operating systems' Aqua, IBM OS/2, Microsoft Windows (e.g., Aero, Metro, etc.), Unix X-Windows, web interface libraries (e.g., ActiveX, Java, JavaScript, AJAX, HTML, Adobe Flash, etc.), or the like.

In some embodiments, computer system 702 may implement web browser 736 stored program component. Web browser 736 may be a hypertext viewing application, such as Microsoft Internet Explorer, Google Chrome, Mozilla Firefox, Apple Safari, etc. Secure web browsing may be provided using HTTPS (secure hypertext transport protocol), secure sockets layer (SSL), Transport Layer Security (TLS), etc. Web browsers may utilize facilities such as AJAX, DHTML, Adobe Flash, JavaScript, Java, application programming interfaces (APIs), etc. In some embodiments, computer system 702 may implement mail server 738 stored program component. Mail server 738 may be an Internet mail server such as Microsoft Exchange, or the like. Mail server 738 may utilize facilities such as ASP, ActiveX, ANSI C++/C#, Microsoft.NET, CGI scripts, Java, JavaScript, PERL, PHP, Python, WebObjects, etc. Mail server 738 may utilize communication protocols such as internet message access protocol (IMAP), messaging application programming interface (MAPI), Microsoft Exchange, post office protocol (POP), simple mail transfer protocol (SMTP), or the like. In some embodiments, computer system 702 may implement mail client 740 stored program component. Mail client 740 may be a mail viewing application, such as Apple Mail, Microsoft Entourage, Microsoft Outlook, Mozilla Thunderbird, etc.

Reply to Reporter

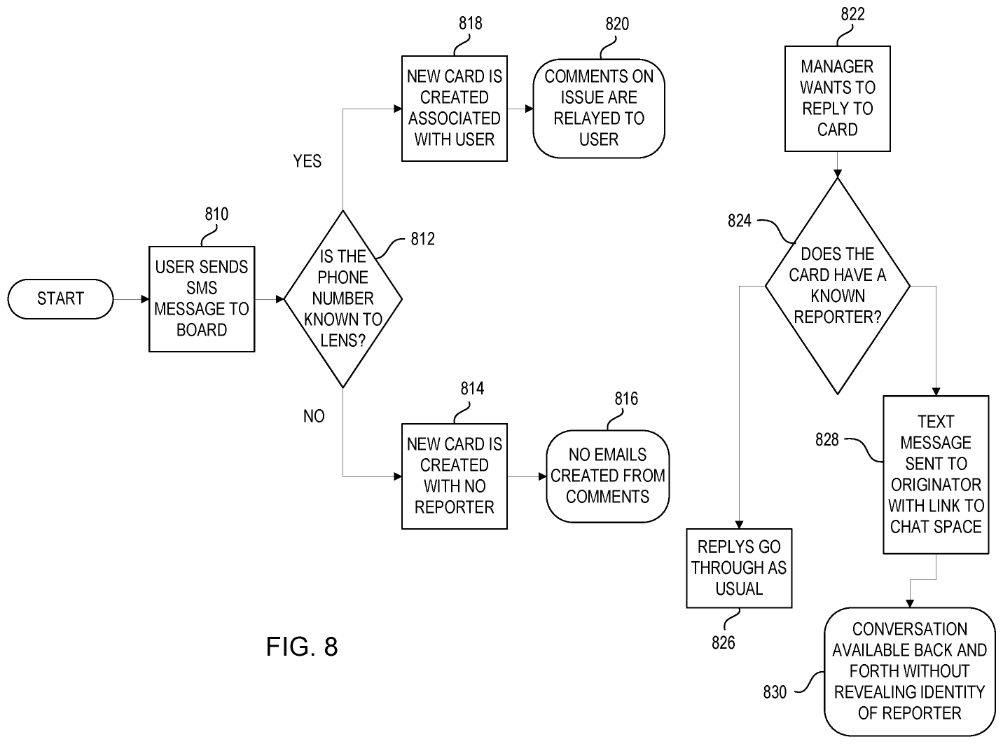

FIG. 8 is a flowchart of the reply to reporter feature which allows for two-way communication between authenticated users and unauthenticated anonymous reporters of issues, without compromising anonymity of the reporters. When a user sends 810 a message to the collaboration engine 1086 in the processor 1080 (FIG. 2), a determination 812 may be made regarding whether the phone number of the phone that sends the SMS message is known to the system. If the phone is not known, a new card may be created 814 without a reporter recorded in the card and no emails are generated 816 from comments. If a determination 812 is made that the phone is known, a new card may be created 818 associated with the known user and comments on the issue can be relayed 820 to the user.

As illustrated on the right side of FIG. 8, when a manager wants to reply 822 to a card, a determination 824 may be made regarding whether the card has a known reporter. If the card has a known reporter, replies go through 826 as usual. If the card does not have a known reporter, a text message may be sent 828 to the originator of the card with a link to a chat space that the originator of the card can access so that an anonymous conversation 830 can take place, if the originator is willing to chat with the manager.

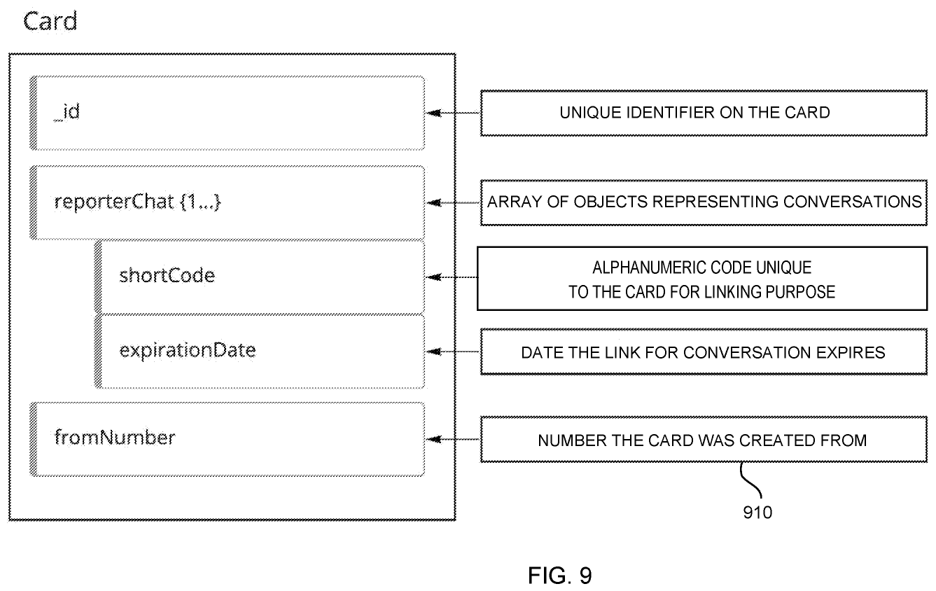

An exemplary data structure of a card created by operations 814 and 818 is illustrated in FIG. 9. Regardless of whether a card has a known reporter, the phone number 910 of the phone that sent an SMS message to the system is recorded in the card along with the other information illustrated in FIG. 9. As a result, when a manager wants to request an anonymous conversation 830 with the originator of the card, a text message can be sent by the system to the phone number 910 to provide a link to an anonymous chat room.

WalkRounds

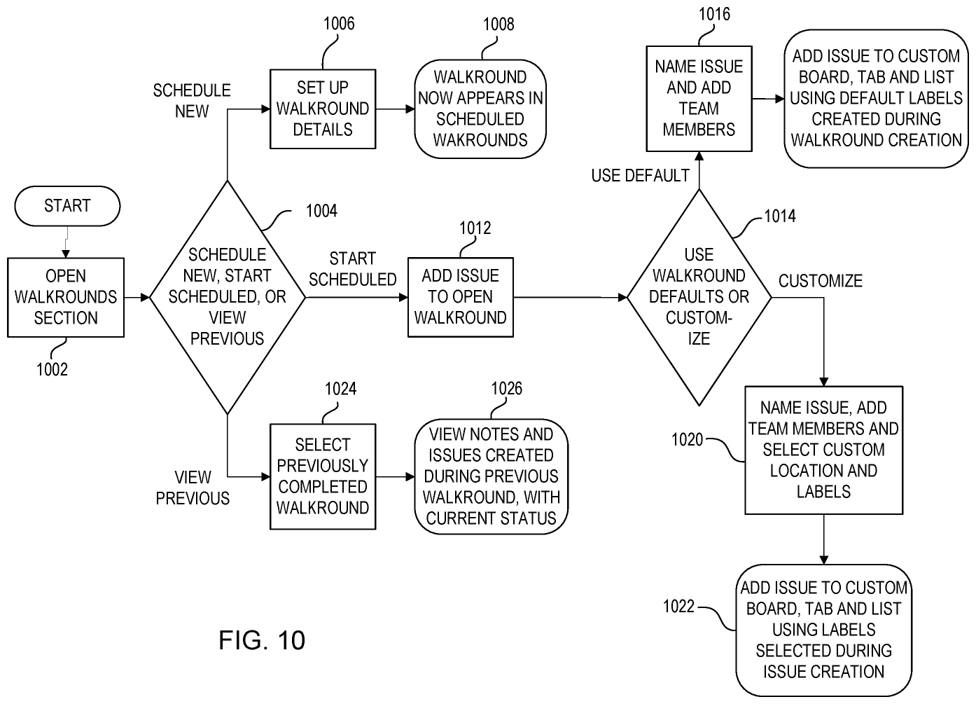

FIG. 10 is a flowchart of a method of scheduling Leadership WalkRounds and facilitating entry of issues found and reported during a walkround. Upon opening 1002 the walkrounds section, a determination 1004 may made regarding whether the user wants to schedule a new walkround, start a scheduled walkround, or view a previous walkround. To schedule a new walkround, the system sets up details 1006. The details may include a name for the walkround, frequency with which the walkround occurs, a time of the walkround, default notes, participants, default location, and labels for issues that will be created during the walkround. After the walkround has been set up, the walkround can appear 1008 in a list of scheduled walkrounds.

If a determination 1004 is made to start a scheduled walkround, the walkround is started 1010, e.g., based on the time or by selecting the walkround from the list of Scheduled walkrounds. During a scheduled walkround, an issue can be added 1012. A determination 1014 may be made regarding to whether to use default label(s) created during setup or make a customized entry. Using default label(s), the user only needs to enter 1016 the name of an issue and add team members involved in the issue. The system then adds 1018 the issue to the board, tab and list with the label(s) set as the defaults when the walkround was created.

In an embodiment, issues may be entered using a customized data entry screen. In addition to the issue name and team members, the user may select 1020 a custom location and label(s). When a customized entry is added 1022 to the board, tab and list, the label(s) selected during issue creation are stored.

As noted above, one of the options that may be selected in the determination 1004 is to view 1024 a previously completed walkround. When this option is selected, a user a user may view 1026 notes and see all issues that were created during that walkround, as well as the current status of those issues.

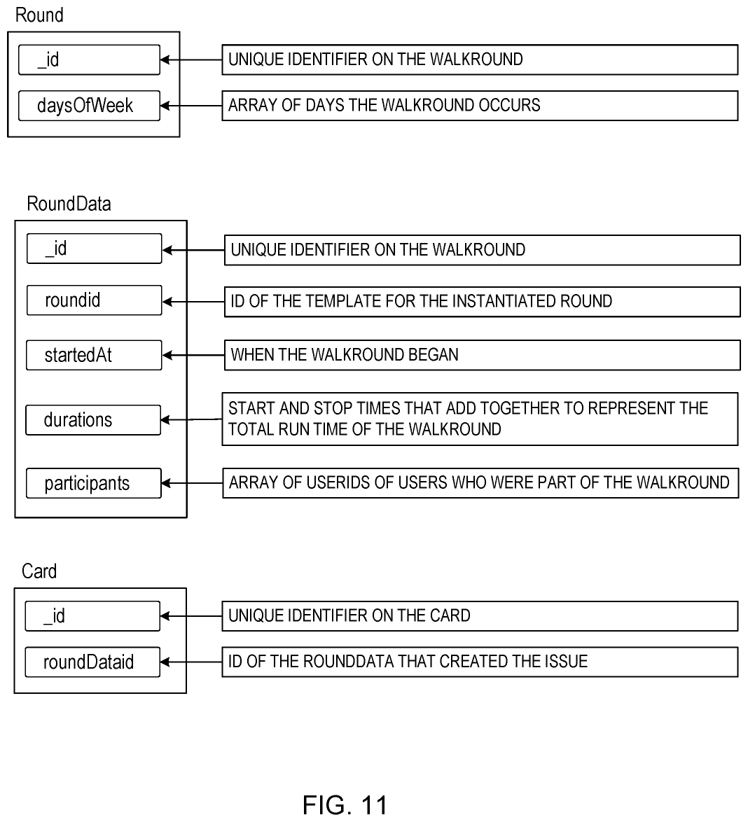

To accommodate the scheduling, recording and viewing of issues noted during walkrounds, the data structure in FIG. 11 may be used. In scheduling a walkround, an array 1102 of days that the walkround occurs is entered. Each occurrence of a walkround may include a beginning 1104 and durations 1106 of starts and stops during the walkround, as well as participants 1108. In addition to other information about an issue that is recorded on a card, a card created during a walkround includes an ID 1110 of the RoundData that created the issue.

The above description has described embodiments of the invention with different functional units and processors. However, any suitable distribution of functionality between different functional units, processors or domains may be used without detracting from the invention. For example, functionality illustrated to be performed by separate processors or controllers may be performed by the same processor or controller. References to specific functional units are only intended as examples of suitable means or mechanisms for providing the described functionality, rather than being indicative of a strict logical or physical structure or organization.

Various embodiments of the invention provide systems and methods for enabling effective communications among several groups within an organization. One exemplary organization is a hospital, and using the system and method of the invention in that setting enables effective communication among various groups of healthcare service providers and administrators. The above described method enables generation of a personalized and contextual summary of a multimedia communication session that may also be thought of as a conference session or a webinar. The summary is generated based on individual needs, topics, roles, or participants by dynamically generating relevant meta-data along with content time stamp by using a semantic analyzer and a voice analyzer.

The specification has also described systems and methods for building contextual highlights for conferencing or communication systems. The illustrated steps are set out to explain the exemplary embodiments shown, and it should be anticipated that ongoing technological development may change how particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments.

Furthermore, one or more computer-readable storage media may be utilized in implementing embodiments consistent with the present disclosure. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., be non-transitory. Examples include random access memory (RAM), read-only memory (ROM), volatile memory, nonvolatile memory, hard drives, CD ROMs, DVDs, flash drives, disks, and any other known physical storage media.

A description has been provided with particular reference to preferred embodiments thereof and examples, but it will be understood that variations and modifications can be effected within the spirit and scope of the claims which may include the phrase “at least one of A, B and C” as an alternative expression that means one or more of A, B and C may be used, contrary to the holding in Superguide v. DIRECTV, 358 F3d 870, 69 USPQ2d 1865 (Fed. Cir. 2004).

What is claimed is:

1. A healthcare data processing system communicating with user devices via a network, comprising:
a communications interface communicating with the user devices via the network; and
a processor configured to
receive input data regarding a healthcare issue from one of the user devices via the network;
determine how to process the input data based on at least one of an identity of the user device and categorization of the input data; and
create a new record in a database of healthcare issues in a healthcare facility where the user device is located when the input data is received, the new record containing the input data and at least one of an identifier associated with the user device and a label associated with the input data.

2. The healthcare data processing system according to claim 1, wherein the processor is further configured to
determine whether the database stored the identifier associated with the user device prior to said creating of the new record,
store in the new record a user identifier when the identifier associated with the user device was stored in the database prior to creation of the new record, and
store in the new record the identifier associated with the user device without any user identifier when the identifier associated with the user device was not stored in the database prior to the creation of the new record.

3. The healthcare data processing system according to claim 2, wherein the processor is further configured to send a request for communication regarding the healthcare issue to the user device when the identifier associated with the user device was not stored in the database prior to creation of the new record

4. The healthcare data processing system according to claim 3, wherein the request for communication regarding the healthcare issue includes a link to an anonymous chat space.

5. The healthcare data processing system according to claim 1,
further comprising a display, and
wherein the processor is further configured to
decide whether to schedule a new walkround by a supervisor, start a scheduled walkround by the supervisor, or view a selected previous walkround by the supervisor,
schedule the new walkround when the new walkround is to be scheduled;
after the scheduled walkround is started, receive and determine how to process the input data, then create the new record, and
output on the display at least one previous walkround by the supervisor for selection to view identified healthcare issues in the database having data recorded during the selected previous walkround.

6. The healthcare data processing system according to claim 5, wherein the processor is further configured to store, when the new walkround is to be scheduled, walkround details in the database, including at least one of a name of the new walkround, frequency of occurrence of the new walkround, a time of the new walkround, default notes, default participants, default location, and default labels for healthcare issues identified during the new walkround.

7. The healthcare data processing system according to claim 5, wherein the processor is further configured to determine how to process the input data by inquiring of the supervisor whether to use default labels, or to receive at least one custom label, for a healthcare issue identified during the scheduled walkround.

8. The healthcare data processing system according to claim 7, wherein the processor is further configured to store in the new record, when default labels are selected by the supervisor, an issue name and at least one identifier of a team member related to the healthcare issue identified during the scheduled walkround.

9. The healthcare data processing system according to claim 7, wherein the processor is further configured to store in the new record, when at least one custom label is selected by the supervisor, the at least one custom label, an issue name and at least one identifier of a team member related to the healthcare issue identified during the scheduled walkround.

10. The healthcare data processing system according to claim 5, wherein the processor is further configured to output on the display, after the selected previous walkround is selected by the supervisor, notes about at least one of the identified healthcare issues in the database having data recorded during the selected previous walkround and current status of the at least one of the identified healthcare issues.

11. A method of communicating between healthcare individuals, comprising:
receiving input data regarding a healthcare issue from a user device via a network;
determining how to process the input data based on at least one of an identity of the user device and categorization of the input data; and
creating a new record in a database of healthcare issues in a healthcare facility where the user device is located when the input data is received, the new record containing the input data and at least one of an identifier associated with the user device and a label associated with the input data.

12. The method according to claim 11,
further comprising determining whether the database stored the identifier associated with the user device prior to said creating of the new record, and
wherein said creating of the new record in the database includes storing a user identifier when said determining determined that the identifier associated with the user device was stored in the database prior to said creating of the new record and storing the identifier associated with the user device without any user identifier when said determining determined that the identifier associated with the user device was not stored in the database prior to said creating of the new record.

13. The method according to claim 12, further comprising sending comments on the healthcare issue indicated by the input data to the user device only when the identifier associated with the user device was stored in the database prior to said creating of the new record.

14. The method according to claim 12, further comprising sending a request for communication regarding the healthcare issue to the user device when said determining determined that the identifier associated with the user device was not stored in the database prior to said creating of the new record.

15. The method according to claim 14, wherein the request for communication regarding the healthcare issue includes a link to an anonymous chat space.

16. The method according to claim 11, further comprising prior to said receiving:
deciding whether to schedule a new walkround by a supervisor, start a scheduled walkround by the supervisor, or view a selected previous walkround by the supervisor;
scheduling the new walkround when said deciding decided that the new walkround is to be scheduled;
performing said receiving, said determining and said creating when said deciding decided that the scheduled walkround was to be started; and
displaying at least one previous walkround by the supervisor for selection to view identified healthcare issues in the database having data recorded during the selected previous walkround.

17. The method according to claim 16, further comprising, when said deciding decided that the new walkround is to be scheduled, storing walkround details in the database, including at least one of a name of the new walkround, frequency of occurrence of the new walkround, a time of the new walkround, default notes, default participants, default location, and default labels for healthcare issues identified during the new walkround.

18. The method according to claim 16, wherein, when said deciding decided that the scheduled walkround was to be started, said determining determines how to process the input data by inquiring of the supervisor whether to use default labels, or to receive at least one custom label, for a healthcare issue identified during the scheduled walkround.

19. The method according to claim 18, wherein, when default labels are selected by the supervisor, said creating the new record stores an issue name and at least one identifier of a team member related to the healthcare issue identified during the scheduled walkround.

20. The method according to claim 18, wherein, when at least one custom label is selected by the supervisor, said creating the new record stores the at least one custom label, an issue name and at least one identifier of a team member related to the healthcare issue identified during the scheduled walkround.
